(19) United States
(12) Patent Application Publication
(10) Pub. No.: US 2021/0045654 A1
(43) Pub. Date: Feb. 18, 2021

(54) MEASUREMENT APPARATUS, MEASUREMENT METHOD AND ELECTRONIC DEVICE FOR MEASURING ENERGY EXPENDITURE OF INDIVIDUAL

(71) Applicants: GETWELL EXERCISE & HEALTH TECHNOLOGY CO. LTD., SHENZHEN, GUANGDONG (CN); GETWELL HEALTH TECHNOLOGY (WUHU) CO. LTD., WUHU, ANHUI (CN)

(72) Inventor: GONG ZHANG, MANITOBA (CA)

(21) Appl. No.: 16/098,540

(22) PCT Filed: Jan. 5, 2017
(86) PCT No.: PCT/CN2017/070338

§ 371 Date: Nov. 2, 2018

Related U.S. Application Data
(60) Provisional application No. 62/346,723, filed on Jun. 7, 2016.

(51) Int. Cl. A61B 5/083; A61B 5/1455; A61B 5/053; A61B 5/0205; A61B 5/145; A61B 5/00; A61B 5/029

(52) U.S. Cl. CPC A61B 5/0833; A61B 5/14552; A61B 5/0531; A61B 5/02055; A61B 5/02416; A61B 5/4519; A61B 5/029; A61B 2560/0252; A61B 2562/0271; A61B 5/14546

(57) ABSTRACT

A measurement apparatus (1, 2, 3, 71), a measurement method and an electronic device (7) for measuring the energy expenditure of an individual. The measurement apparatus (1, 2, 3, 71) comprises: a near-infrared unit (10) for emitting near-infrared rays to muscle tissue of an individual, so as to determine muscle oxygenation value of the individual by reflection of the near-infrared rays by the muscle tissue; an electrode array (20) for measuring conductivity of skin of the individual; and a control unit (30) operably connected to the near-infrared unit (10) and the electrode array (20), to control activation of the near-infrared unit (10) and the electrode array (20), and to obtain muscle oxygenation value and conductivity, so as to determine energy expenditure of the individual based on muscle oxygenation value and conductivity and skin temperature of the individual and ambient temperature. The measurement apparatus (1, 2, 3, 71), the measurement method and the electronic device (7) for measuring energy expenditure of an individual, at least can measure both activity-related energy expenditure and rest energy expenditure..

### TECHNICAL FIELD

The present application relates to the field of sports health, and more particularly to a measurement apparatus, a measurement method and an electronic device for measuring energy expenditure of an individual.

### BACKGROUND

In the field of sports health, measuring energy expenditure of an individual is very important for energy balance of an individual, especially for individuals affected by metabolism related chronic diseases (e,g., diabetes, cardiovascular disease, etc.). In the prior art, measurement apparatuses comprising activity sensors such as acceleration sensors are typically used to measure the energy expenditure of an individual; however, these activity sensors are generally unable to measure rest energy expenditure that accounts for more than 80% of the total energy expenditure of the body.

Accordingly, an apparatus capable of measuring the energy expenditure comprising rest energy expenditure is desired.

### SUMMARY

The brief summary of the present application will be presented below to provide basic understanding of some aspects of the invention. It should be understood that the summary is not an exhaustive one of the present application, It is not intended to define a key or important part of the present application, or to limit the scope of the present application. The purpose is just to present some concepts in a simplified form, as a preface of detailed descriptions described subsequently.

In view of the above-described deficiencies in the prior art, it is one of the objects of the present application to provide a measurement apparatus, a measurement method and an electronic device for measuring energy expenditure of an individual, to at least overcome the deficiencies in the prior art.

An embodiment of the present application provides a measurement apparatus, comprising: a near-infrared unit for emitting near-infrared rays into muscle tissue of an individual, to determine muscle oxygenation value of the individual by reflection of the near-infrared rays by the muscle tissue; an electrode array for measuring conductivity of skin of the individual; and a control unit operatively coupled to the near-infrared unit and the electrode array, to control activation of the near-infrared unit and the electrode array, and to obtain the muscle oxygenation value and the conductivity, so as to determine energy expenditure of the individual based on the muscle oxygenation value and the conductivity and skin temperature of the individual and ambient temperature.

Another embodiment of the present application provides a method for measuring energy expenditure of an individual, comprising: emitting near-infrared rays into muscle tissue of the individual, to determine a muscle oxygenation value of the individual by reflection of the near-infrared rays by the muscle tissue; measuring conductivity of the skin of the individual; and obtaining the muscle oxygenation value and the conductivity, so as to determine the energy expenditure of the individual based on the muscle oxygenation value and the conductivity and skin temperature of the individual and ambient temperature,

Yet another embodiment of the present application provides an electronic device for measuring energy expenditure of an individual, comprising: a measurement apparatus and an electronic apparatus capable of communicating with the measurement apparatus. The measurement apparatus comprises: a near-infrared unit for emitting near-infrared rays into muscle tissue of an individual, to determine a muscle oxygenation value of the individual by reflection of the near-infrared rays by the muscle tissue; an electrode array for measuring conductivity of the skin of the individual; and a control unit operatively coupled to the near-infrared unit and the electrode array, to control activation of the near-infrared unit and the electrode array, and to obtain and transmit the muscle oxygenation value and the conductivity, The electronic apparatus receives the muscle oxygenation value and the conductivity from a control unit of the measurement apparatus, and determines the energy expenditure of the individual based on the muscle oxygenation value and the conductivity and skin temperature of the individual and ambient temperature.

The measurement apparatus and the measurement method and the electronic device for measuring rest energy expenditure of an individual according to the present application have at least one of the following, advantages: being able to measure both activity-related energy expenditure and rest energy expenditure; being able to measure energy expenditure in a non-invasive manner; easy to wear on the body, and being able to achieve real-time measurement of the energy expenditure.

### BRIEF DESCRIPTION OF THE DRAWINGS

One or more embodiments are illustratively described in combination with drawings in the corresponding accompanying drawings, and these illustrative descriptions should not be construed as limiting the embodiments. Elements with like reference numerals represent similar elements, Drawings in the accompanying drawings are not drawn to scale unless specifically stated otherwise.

FIG. 1 is a block diagram showing an exemplary structure of a measurement apparatus according to a first embodiment of the present application.

FIG. 2 is a block diagram showing an exemplary structure of a measurement apparatus according to a second embodiment of the present application,

FIG. 3 is a block diagram schematically showing an exemplary structure of a near-infrared unit according to the first embodiment and the second embodiment of the present application.

FIG. 4 is a graph showing the relationship between the extinction coefficient of near-infrared rays and near-infrared rays wavelength for oxyhemoglobin and deoxyhemoglobin.

FIG. 5 shows a block diagram of an exemplary structural of a measurement apparatus according to a third embodiment of the present application,

FIG. 6 is a flow chart showing an exemplary process of a measurement method according to an embodiment of the present application,

FIG. 7 is a block diagram showing an exemplary structure of an electronic device according to an embodiment of the present application.

### DETAILED DESCRIPTION OF THE EMBODIMENTS

In order to make the objects, technical solutions and advantages of the present application clearer, some embodiments of the present application will be further described in detail below in combination with the accompanying drawings and embodiments. It should he understood that the specific embodiments described herein are merely illustrative of the present application, and not intended to limit the present application.

In the field of sports health, it is desirable to he able to easily obtain energy expenditure of an individual in various activity states, thereby realizing judgment and tracking of human health conditions based on energy expenditure. Therefore, there exists a need in the art for a measurement apparatus capable of measuring energy expenditure in various activity states, particularly for those with easy installation on an individual's body part, such as the arm or leg of a human body.

FIG. 1 is a block diagram showing an exemplary structure of a measurement apparatus according to a first embodiment of the present application. As shown in FIG. 1, the measurement apparatus 1 comprises: a near-infrared unit 10 for emitting near-infrared rays into muscle tissue of an individual, to determine a muscle oxygenation value of the individual by reflection of the near-infrared rays by the muscle tissue; an electrode array 20 for measuring conductivity of the skin of the individual; and a control unit 30 operatively coupled to the near-infrared unit 10 and the electrode array 20, to control activation of the near-infrared unit 10 and the electrode array 30, and to obtain and transmit the muscle oxygenation value and the conductivity, so as to determine the energy expenditure of the individual based on the muscle oxygenation value and the conductivity and skin temperature of the individual and ambient temperature.

According to the present application, the individual who served as the measurement object of the measurement apparatus may be, for example, a living body such as a human body or an animal body. The above-described measurement apparatus 1 according to the first embodiment of the present application facilitates mounting on a body part of an individual, such as an arm or a kg, thereby achieving measurement of the individual's energy expenditure through measuring muscle oxygenation value of the body part and conductivity of the skin at the body part. For example, the measurement apparatus can be mounted selectively on the appropriate body part of the individual depending on the type of sports the individual is engaged in, for example, the measurement apparatus can be mounted on a body part that is primarily used by the individual for sports. However, the present disclosure is not limited thereto, and those skilled in the art can understand that the specific mounting site of the measurement apparatus can also be determined according to actual needs. For example, if the individual is running, the measurement apparatus can be mounted on the individual's leg or mounted on the arm or other sites.

The near-infrared unit 10 of the above-described measurement apparatus 1 according to the first embodiment of the present application can, for example, utilize near infrared rays spectroscopy (NIRS) to evaluate muscle oxygenation values of an individual during various states including a rest state and a motion state. Determining a muscle oxygenation value of an individual with NIRS is performed in a non-invasive manner.

Since near-infrared rays can relatively easily penetrate muscle tissue of the human body, and oxyhemoglohin and deoxyhemoglobin for determining muscle oxygenation values have different absorption rates for near-infrared rays in different wavelength ranges, and based on this, near-infrared unit 10 can determine the muscle oxygenation value of the individual by emitting near-infrared rays to the muscle tissue of the individual and by reflection of the near-infrared rays by the muscle tissue.

According to the first embodiment of the present application, the electrode array 20 is used for measuring the conductivity of the skin of an individual, and the electrode array 20 can achieve a measure of skin conductivity in any manner known in the art, and the specific measurement manner of which will not be repeated herein. For example, the electrode array according to the first embodiment of the present application may be implemented with a muscle electromyography sensor array, hut the present application is not limited thereto, any other form of electrode array can be used to implement electrode array in a measurement apparatus according to the present application, as long as these electrode arrays are capable of measuring conductivity of the skin.

In the first embodiment of the present application, control unit 30 is operatively coupled to the near-infrared unit 10 and electrode array 20 for controlling activation of the near-infrared unit 10 and the electrode array 20, and for receiving muscle oxygenation values and conductivity from the near-infrared unit 10 and the electrode array 20. Control unit 30 can be implemented with a combinational logic controller of the prior art. However, the present disclosure is not limited thereto, and the control unit 30 may be implemented with, for example, a microprogram controller (for example, a CPU).

According to an embodiment of the present application, the control unit 30 may be configured to control the near-infrared unit 10 and the electrode array 20 to periodically activate the near-infrared unit and the electrode array.

According to the present application, after obtaining muscle oxygenation value of the individual and conductivity of the skin, the control unit 30 may transmit the muscle oxygenation value and the conductivity to an external apparatus such as a mobile terminal, so that the external apparatus calculates the energy expenditure of the individual based on the muscle oxygenation value and the conductivity of the skin and skin temperature of the individual and ambient temperature. However, the present disclosure is not limited thereto, for example, in the case where the control unit 30 is implemented with a controller having a calculation function, the calculation of the individual's energy expenditure based on the muscle oxygenation value and the conductivity of skin as well as skin temperature of the individual and ambient temperature can also be performed by the control unit 30.

According to the present application, skin temperature of an individual and ambient temperature may be obtained by the control unit through communication with a temperature sensor, for example located outside the measurement apparatus, or the measurement apparatus may also comprise a temperature sensor to measure skin temperature of the individual and ambient temperature.

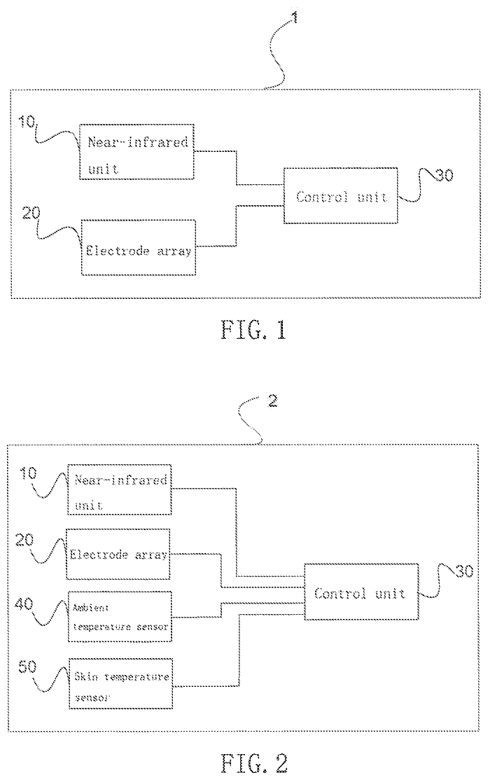

FIG. 2 is a block diagram showing an exemplary structure of a measurement apparatus according to a second embodiment of the present application,

As shown in FIG. 2, in addition to comprising the near-infrared unit 10, the electrode array 20, and the control unit 30, similar to the measurement apparatus i of FIG. 1, the measurement apparatus 2 may further comprise: an ambient temperature sensor 40 operatively coupled to the control unit 30, and the ambient temperature sensor 40 is configured to measure ambient temperature and transmit the ambient temperature to the control unit 30; and a skin temperature sensor 50 operatively coupled to the control unit 30, and the skin temperature sensor 50 is configured to measure the skin temperature of the individual and transmit the measured skin temperature to the control unit 30.

The measurement apparatus 2 according to the second embodiment of the present application can measure the ambient temperature and the skin temperature with any existing ambient temperature sensors and skin temperature sensors, and the specific measurement manner thereof will not be repeated herein.

The control unit 30 of the measurement apparatus 2 according to the second embodiment of the present application may also be configured to control the ambient temperature sensor 40 and the skin temperature sensor 50 to activate the ambient temperature sensor 40 and the skin temperature sensor 50, for example, the control unit 30 may control periodically to activate the ambient temperature sensor 40 and the skin temperature sensor 50.

FIG. 3 is a block diagram schematically showing an exemplary structure of the near-infrared unit 10 according to the first embodiment and the second embodiment of the present application.

As shown in FIG. 3, the near-infrared unit 10 comprises: a near-infrared rays emitter 101 for emitting a plurality of sets of near-infrared rays with different wavelengths to muscle tissue of an individual respectively; and a near-infrared rays receiver 102 for receiving each set of near-infrared rays of the plurality of sets of near-infrared rays reflected from the muscle tissue; and a processing module 103 for determining an individual's hemoglobin value and myoglobin value based on the plurality of sets of reflected rays received by the near-infrared receiver, and determining muscle oxygenation value of the muscle tissue based on the hemoglobin value and the myoglobin value.

According to the present application, the near-infrared rays emitter 101 may be, for example, an LED light capable of emitting near-infrared rays, but the present application is not limited thereto, and those skilled in the art shall understand that the near-infrared rays emitter 101 according to the present application may also be other emitters capable of emitting near-infrared rays. According to the present application, the near-infrared rays receiver 102 can be implemented, for example, with a photodiode.

According to the present application, the processing module 103 may be further configured to be directed to each set of near-infrared rays to determine attenuation value of the near-infrared rays according to emission current of the near-infrared rays emitter and the reception current of the near-infrared rays receiver, and determine oxyhemoglobin and deoxyhemoglobin of the muscle tissue based on attenuation value of the plurality of sets of near-infrared, thereby determining muscle oxygenation value of the individual based on the determined oxyhemoglobin and deoxyhemoglobin.

According to an embodiment of the present application, the processing module 30 may determine the oxyhemoglobin value and the deoxyhemoglobin value, for example, according to Lambert-Beers law, and more specifically may determine the oxyhemoglobin value and deoxyhemoglobin, for example, with the following equation (1):

$$A ( λ ) = ln \frac{{I}_{0} ( λ )}{I ( λ )} = ( {C}_{0} + {C}_{1} λ ) + L [ {C}_{hhb} {ɛ}_{hhb} ( λ ) + {C}_{{hb}_{o}} {ɛ}_{{hb}_{o}} ( λ ) ] ln 10 \qquad ( 1 )$$

wherein, A is the attenuation value of near-infrared rays after incoming on the muscle tissue, I₀ is the input light intensity, I is the reflected light intensity, C₀+C₁λ is the attenuation other than hemoglobin and water, and L is the distance of the near-infrared rays from the transmitting end to the receiving end (for example, the near-infrared receiver 102 can be disposed within a range of 10 mm to 20 mm from the near-infrared rays emitter 101), and the G_{hhb} and C_{hbo} are deoxyhernoglobin density (also referred to as deoxyhemoglobin value) and oxyhemoglobin density (also known as oxyhemoglobin value), respectively, ε_{hhb}, ε_{hbo} are the extinction coefficients of the deoxyhemoglobin and the oxyhemoglobin for near-infrared rays, respectively.

The near-infrared unit 10 can calculate the attenuation value A of the near-infrared rays, for example, based on the emission current of the LED light which emits the near-infrared rays and the reflected current I_{PD} formed by the reflected rays received by the near-infrared receiver, However, the present disclosure is not limited thereto, and the attenuation value A of the near-infrared rays may be calculated by other methods known in the art.

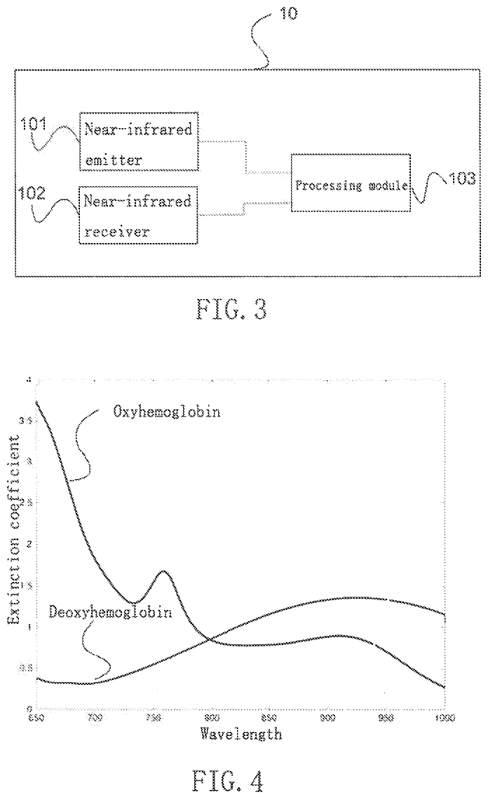

FIG. 4 is a graph showing the relationship between extinction coefficients ε_{hbo}, ε_{hhb} of the oxyhemoglobin and deoxyhemoglobin for the near-infrared rays and the near-infrared rays wavelength. That is, the extinction coefficients ε_{hbo} and ε_{hhb} of the above-described oxyhemoglobin and deoxyhemoglobin for near-infrared rays can be determined by the wavelength of near-infrared rays emitted from the near-infrared rays emitter 101.

The processing module 130 may obtain the deoxyhemoglohin density C_{hhb} and oxyhemoglobin C_{hbo}, according to the attenuation of at least four different wavelengths and by solving the optimal value with a nonlinear optimization method based on the above-described equation (1).

After obtaining the oxyhemoglobin density and the deoxyhemoglobin density, the processing module 130 can calculate the muscle oxygenation value based on the oxyhernoglobin density and the deoxyhemoglobin density, for example, the processing module 130 can calculate the muscle oxygenation value SₘO², based on, for example, the following equation (2):

$${S}_{m} {O}^{2} = \frac{Chbo}{Chbo + Chhb} \qquad ( 2 )$$

wherein, C_{hhb} is the deoxyhemoglobin density and C_{hbo} is the oxyhemoglobin density.

According to the present disclosure, the near-infrared rays emitter 101 of the near-infrared unit 10 is preferably configured to emit near-infrared rays with wavelengths of 660 nm, 730 nm, 810, 850 nm and 940 nm.

According to another embodiment of the present application, the processing module 130 may determine the hemoglobin value and the myoglobin value of the individual through the plurality of sets of the reflected rays received by the near-infrared rays receiver 102, and determine the muscle oxygenation value of the muscle tissue based on the hemoglobin value and the myoglobin value. For example, the processing module 130 can calculate the muscle oxygenation value SₘO² by the following equation (3):

SₘO²=Δ(C_{hbo}+O²Mb—(C_{hhb}+HMb)) (3)

wherein, C_{hhb} is the deoxyhemoglobin density, C_{hbo} is the oxyhemoglobin density, O²MB is an oxymyoglobin density, and HMb is an deoxymyoglobin density,

The oxymyoglohin density O²Mb and the deoxyhemoglobin density HMb can be obtained, for example, with any method in the prior art, based on the oxyhemoglobin density and the deoxyhemoglobin density, The specific obtaining manner is well known in the art and will not be repeated herein.

According to an embodiment of the present application, the control unit 30 may be further configured to calculate the radiant heat which the individual radiates to the outside during oxygen consumption, according to the conductivity of skin, the difference between skin surface temperature of the individual and ambient temperature acquired from the electrode array 20, and determine cardiac output of the individual based on the radiant heat, heart rate and muscle oxygenation value of the individual, thereby determining oxygen consumption of the individual based on cardiac output and muscle oxygenation value of the individual. The individual's skin surface temperature and the ambient temperature may be obtained by communicating with an external apparatus located outside the measurement apparatus, or in the case where the measurement apparatus 2 comprises the skin temperature sensor 50 and the ambient temperature sensor 40 as shown in FIG. 2, the individual's skin temperature and the ambient temperature are obtained from the skin temperature sensor 50 and the ambient temperature sensor 40, respectively,

The control unit 30 can calculate the amount of heat radiated to the outside by the individual when oxygen is consumed, for example, based on the conductivity measured by the electrode array 20, the difference between the skin surface temperature and the ambient temperature, and the surface skin area of the individual. The area of the individual's surface skin can be obtained according to the height and weight of the individual with any method known in the art.

The amount of heat H radiated by an individual to the outside during oxygen consumption is related to the individual's cardiac stroke volume, heart rate, and the oxygen content of the blood introduced into the tissue (i.e., muscle oxygenation value), while cardiac output is usually calculated through the cardiac stroke volume and the heart rate, so that the cardiac output Q can be determined based on the amount of heat H radiated to the outside, heart rate HR, and muscle oxygenation value SₘO² during oxygen consumption. For example, the cardiac stroke volume SV for determining the final oxygen consumption amount can be determined according to the following equation (4), and is determined according to the following equation (5):

SV=H/C X HR X SₘO² (41)

Q=SV X HR (5)

wherein H is the heat radiated to the outside by the body during oxygen consumption, as described above, which may be determined according to the conductivity, the difference between the skin surface temperature and the ambient temperature measured by the electrode array 20, and the area of the individual's surface skin. The parameter C is a parameter reflecting the characteristics of different individuals, which can be determined according to the gender, height, weight and age of the individual; those skilled in the art shall understand that the parameter C can be determined in advance according to various methods, such as, generating a suitable database according to specific parameters, or using approximation and/or extrapolation method of the previously measured values.

Moreover, the heart rate of the individual for determining the cardiac output Q can be obtained from the external apparatus by communicating with the external apparatus other than the control unit 30 and the measurement apparatus. However, the present disclosure is not limited thereto, and for example, the heart rate of the individual can also be obtained by configuring the measurement apparatus to comprise a heart rate measurement unit.

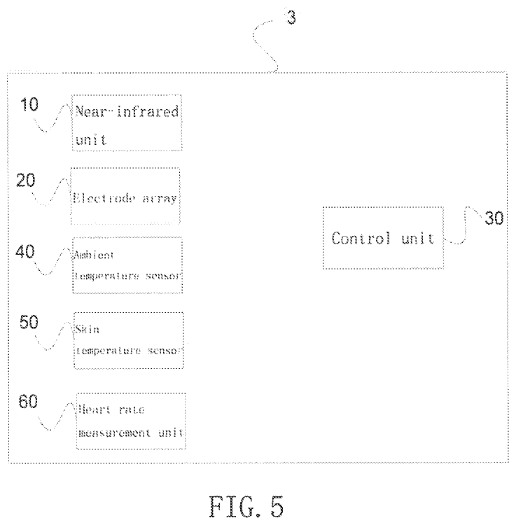

FIG. 5 shows a block diagram of an exemplary structural of a measurement apparatus according to a third embodiment of the present application, As shown in FIG. 5, in addition to comprising the near-infrared unit 10, the electrode array 20, the control unit 30, the ambient temperature sensor 40, and the skin temperature sensor 50, similar to the measurement apparatus 2 of FIG. 2, the measurement apparatus 3 comprises: a heart rate measurement unit 60 operatively coupled to the control unit 30, and the heart rate measurement unit 60 is configured to measure an heart rate of the individual and transmit the measured heart rate to the control unit 30. The heart rate measurement unit 60 can measure the heart rate of the individual in any manner in the prior art, and the specific measurement manner will not be repeated herein.

After the control unit 30 obtains the muscle oxygenation value SₘO² from the near-infrared unit 10 and determines the cardiac output amount Q, the control unit 30 may further determine the oxygen consumption amount VO² based on the muscle oxygenation value SₘO² and the cardiac output amount Q. For example, the control unit 30 can determine the oxygen consumption amount VO² according to the following equation (6) based on the Fick's equation,

VO²=Q×((97-SₘO²)/100×1.34×C_{hhb}×10 (6)

Wherein C_{hhb} is the individual's oxyhemoglobin value, which can be obtained, for example, when the near-infrared unit 10 determines the muscle oxygenation value SₘO².

After determining the oxygen consumption amount, the control unit 30 may further calculate the calorie consumption amount in the process of consuming oxygen, based on the oxygen consumption amount and the weight of the individual. Any method in the prior art can be used to determine calorie consumption amount based on oxygen consumption amount, For example, the calorie consumption amount F can be calculated based on the oxygen consumption amount by the following equation (7).

E=VO²×W×K (7)

Wherein, VO² is the oxygen consumption of the individual; W is the weight of the individual; K is a constant, which can be set by those skilled in the art according to actual conditions, for example, it can be set to 5.

The manners of determining the calorie consumption amount based on the oxygen consumption amount are exemplarily shown above, however, the present application is not limited thereto, and those skilled in the art shall understand that other methods of determining calorie consumption amount based on oxygen consumption amount in the prior art can also be used to determine the calorie consumption amount.

The above embodiment describes that in the case where the control unit 30 is implemented with a controller having an calculation function, the control unit determines the oxygen consumption amount based on the muscle oxygenation value of the individual and the conductivity of the skin, thereby determining the calorie consumption amount. However, the present disclosure is not limited thereto, and those skilled in the art shall understand that the operation of determining the oxygen consumption amount based on the muscle oxygenation value of the individual and the conductivity of the skin can also he performed by the processing module 103 of the near-infrared unit 10. Alternatively, the operation of determining the oxygen consumption amount and further determining the calorie consumption amount based on the muscle oxygenation value of the individual and the conductivity of the skin may also be performed by an external apparatus (for example, a mobile terminal). Similar to the operation of the control unit 30 to determine the oxygen consumption amount based on the muscle oxygenation value of the individual and the conductivity of the skin, thereby determining the calorie consumption amount, the processing for performing the above-described determining operation by the processing module 103 of the near-infrared unit 10 and the external apparatus will not be repeated herein.

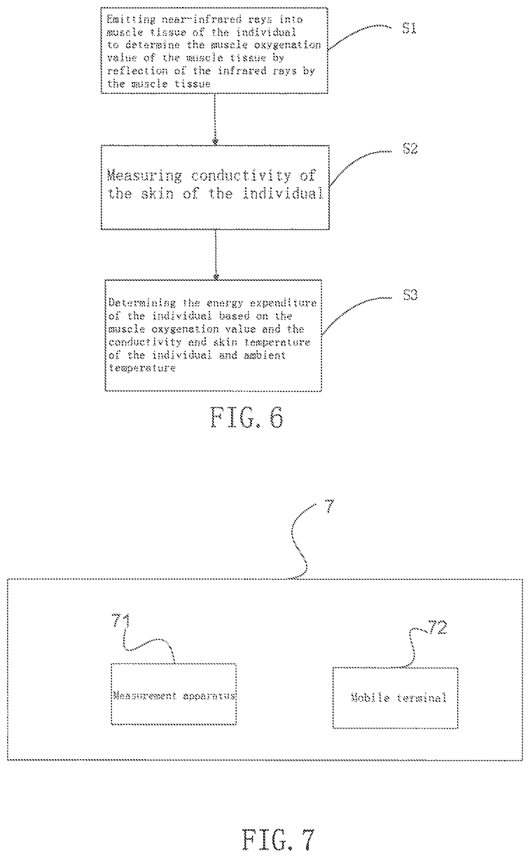

According to the present application, a measurement method for measuring the energy expenditure of an individual is further provided. An exemplary process of the measurement method is described below in conjunction with FIG. 6.

As shown in FIG. 6, the measurement method according to an embodiment of the present application comprises: in step S1, emitting near-infrared rays into muscle tissue of the individual to determine the muscle oxygenation value of the muscle tissue by reflection of the infrared rays by the muscle tissue; in step S2, measuring conductivity of the skin of the individual; and in step S3, determining the energy expenditure of the individual based on the muscle oxygenation value and the conductivity and skin temperature of the individual and ambient temperature. For example, steps S1, S2, S3 may be respectively implemented by performing, fhr example, operations of the near-infrared unit 10, the electrode array 20, and the control unit described with reference to FIG. 1, and a detailed description thereof will be omitted herein.

According to the present application, an electronic device for measuring energy expenditure of an individual is also provided.

FIG. 7 illustrates a block diagram of an exemplary structural of the electronic device according to an embodiment of the present application, As shown in FIG. 7, the electronic device comprises: a measurement apparatus 71 for measuring muscle oxygenation value of an individual and conductivity of the skin; and an electronic apparatus 72 for receiving the muscle oxygenation value and the conductivity from the measurement apparatus 71, and for determining the energy expenditure of the individual based on the muscle oxygenation value and the conductivity.

The measurement apparatus 71 according to the present disclosure may be the measurement apparatus described with reference to FIG. 14. As shown in FIG. 7, the measurement apparatus 71 comprises: a near-infrared unit 711 for emitting near-infrared rays into the muscle tissue of the individual to determine muscle oxygenation value of the muscle tissue by reflection of the infrared rays by the muscle tissue; an electrode array 712 for measuring the conductivity of the skin of the individual; and a control unit 713 operatively coupled to the near-infrared rays emitter and the electrode array to control activation of the near-infrared rays emitter and the electrode array and to obtain and tranmit the muscle oxygenation value and the conductivity to the electronic apparatus 72.

Compared to the prior art, the measurement apparatus and the measurement method and the electronic device for measuring energy expenditure of an individual according to the present application have at least one of the following advantages: being able to measure both activity-related expenditure and rest energy expenditure; being able to measure the energy expenditure in a non-invasive manner; easy to wear on the body, being able to achieve real-time measurement of energy expenditure.

Finally, it should also be noted that in the present disclosure, relational terms such as “first” and “second”, etc., are only used to distinguish one entity or operation from another entity or operation, without necessarily requiring or imply any these actual relationship or order between these entities or operations. Furthermore, the term “comprise(s)” or “include(s)” or any other variants thereof is intended to encompass a non-exclusive inclusion, such that a process, method, item, or device which comprises a plurality of elements includes not only those elements but also other elements which are not specifically listed, or elements that are inherent to such a process, method, item, or device. In the absence of more restrictions, the elements defined by the sentence “comprise(s) a(an) . . . ” do not rule out additional identical elements that may be contained in the process, method, item or device comprising said elements,

While the disclosure has been disclosed in the foregoing description of the embodiments of the present invention, it shall be understood that those skilled in the art can design various modifications, improvements or equivalents of the present disclosure within the spirit and scope of the appended claims. Such modifications, improvements or equivalents should also be considered to be included within the scope of the disclosure.

1. A measurement apparatus, comprising:
a near-infrared unit for emitting near-infrared rays into muscle tissue of an individual, to determine muscle oxygenation value of the individual by reflection of the near-infrared rays by the muscle tissue;
an electrode array for measuring conductivity of skin of the individual; and
a control unit operatively coupled to the near-infrared unit and the electrode array, to control activation of the near-infrared unit and the electrode array, and to obtain the muscle oxygenation value and the conductivity, so as to determine energy expenditure of the individual based on the muscle oxygenation value and the conductivity and skin temperature of the individual and ambient temperature.

2. The measurement apparatus of claim 1, further comprising:
an ambient temperature sensor operatively coupled to the control unit, and the ambient temperature sensor being configured to measure ambient temperature and transmit the ambient temperature to the control unit; and
a skin temperature sensor operatively coupled to the control unit, and the skin temperature sensor being configured to measure skin temperature of the individual, and transmit the skin temperature to the control unit,

3. The measurement apparatus of claim 1, wherein the near-infrared unit comprises:
a near-infrared rays emitter for emitting a plurality of sets of near-infrared rays with different wavelengths to muscle tissue of the individual respectively;
a near-infrared rays receiver for receiving each set of near-infrared rays of the plurality of sets of near-infrared rays reflected from the muscle tissue; and
a processing module for determining hemoglobin value of the individual based on the plurality of sets of reflected rays received by the near-infrared receiver, and determining muscle oxygenation value of the muscle tissue based on the hemoglobin value,

4. The measurement apparatus of claim 3, wherein the processing module is further configured to be directed to each set of near-infrared rays to determine attenuation value of the near-infrared rays according to emission current of the near-infrared rays emitter and the reception current of the near-infrared rays receiver, and determine oxyhemoglobin value and deoxyhemoglobin value of the muscle tissue based on attenuation value of the plurality of sets of near-infrared, thereby determining muscle oxygenation value of the individual based on the determined oxyhemoglobin value and deoxyhemoglobin value.

5. The measurement apparatus of claim 3, wherein the near-infrared rays emitter emits five sets of near-infrared rays with wavelengths of 660 nm, 730 nm, 810, 850 nm and 940 nm, respectively, to the muscle tissue, to facilitate determination of muscle oxygenation value of the muscle tissue based on reflection of each set of near-infrared rays of the five sets of near-infrared rays.

6. The measurement apparatus of claim 1, wherein the control unit is further configured to calculate radiant heat which the individual radiates to the outside during oxygen consumption, according to the conductivity and difference between skin surface temperature of the individual and ambient temperature, and determine cardiac output of the individual based on the radiant heat, heart rate and muscle oxygenation value of the individual, thereby determining oxygen consumption of the individual based on cardiac output and muscle oxygenation value of the individual.

7. The measurement apparatus of claim 6, further comprising:
a heart rate measurement unit operatively coupled to the control unit, and the heart rate measurement unit being configured to measure heart rate of the individual and transmit the measured heart rate to the control unit.

8. The measurement apparatus of claim 6, wherein the control unit is further configured to determine calorie consumption amount based on oxygen consumption amount of the individual.

9. The measurement apparatus of claim 1, wherein the control unit is further configured to control the near-infrared unit and the electrode array to periodically activate the near-infrared unit and the electrode array,

10. A method for measuring energy expenditure of an individual, comprising:
emitting near-infrared rays into muscle tissue of the individual to determine muscle oxygenation value of the muscle tissue by reflection of the infrared rays by the muscle tissue;
measuring conductivity of skin of the individual; and
obtaining the muscle oxygenation value and the conductivity, to determine energy expenditure of the individual based on the muscle oxygenation value and the conductivity and skin temperature of the individual and ambient temperature,

11. An electronic device for measuring energy expenditure of an individual, comprising:
a measurement apparatus which comprises;
a near-infrared unit for emitting near-infrared rays into muscle tissue of the individual, to determine muscle oxygenation value of the individual by reflection of the near-infrared rays by the muscle tissue;
an electrode array for measuring conductivity of skin of the individual; and
a control unit operatively coupled to the near-infrared unit and the electrode array, to control activation of the near-infrared unit and the electrode array, and to obtain and transmit the muscle oxygenation value and the conductivity; and
an electronic apparatus for receiving the muscle oxygenation value and the conductivity from control unit of the measurement apparatus, and for determining energy expenditure of the individual based on the muscle oxygenation value and the conductivity and skin temperature of the individual and ambient temperature.

12. The electronic device of claim 11, wherein the electronic device is a mobile device, particularly a mobile terminal.
